(19) United States
(12) Patent Application Publication
(10) Pub. No.: US 2025/0099088 A1
(43) Pub. Date: Mar. 27, 2025

(54) CARRIAGE DEVICE FOR A SURGICAL INSTRUMENT

(71) Applicant: KARL STORZ SE & Co. KG, Tuttlingen (DE)

(72) Inventor: Uwe WITTKE, Tuttlingen (DE)

(21) Appl. No.: 18/891,205

(22) Filed: Sep. 20, 2024

(30) Foreign Application Priority Data
Sep. 21, 2023 (DE) 10 2023 125 617.3

(51) Int. Cl. A61B 17/00

(52) U.S. Cl. CPC A61B 17/00; A61B 2017/00477

(57) ABSTRACT

A carriage device for a surgical instrument with at least two instruments, with a kinematic unit configured for actuation, with a first carriage module configured for coupling to a first instrument, and with a second carriage module configured for coupling to a second instrument. The two carriage modules are movable relative to each other and can be individually coupled to the kinematic unit in order to use the first instrument or the second instrument. The first carriage module and the second carriage module can be fixed to the carriage base when they are not in use. A surgical instrument is also provided with two instruments, having a carriage device.

### CROSS REFERENCE TO RELATED APPLICATIONS

This application claims the benefit of priority under 35 U.S.C. § 119 of German Application 10 2023 125 617.3, filed Sep. 21, 2023, the entire contents of which are incorporated herein by reference.

### TECHNICAL FIELD

The present invention relates to a carriage device for a surgical instrument with at least two instruments and to a surgical instrument with two instruments, having a carriage device.

### BACKGROUND

Surgical instruments are used for different applications. For example, they can be used in the area of minimally invasive surgery and have a surgical tool. Surgical instruments can be configured as resectoscopes and have a working element that is configured, for example, to guide the surgical tool, such as an electrode.

Resectoscopes are particularly well known for urological or, for example, gynecological applications. The working element with the electrode can be inserted into the patient's body through the urethra to a surgical site. At one distal end (i.e., remote from the user), for example, the electrode has a cutting loop that can be used to ablate tissue. The ablated tissue can be removed again through a drain channel by means of an irrigation liquid that is guided through an irrigation channel or inflow channel to the surgical site. The inflow and drain channels enable continuous rinsing, which also serves to keep a viewing window clear to ensure a perfect endoscopic view at all times.

Surgical instruments generally have a handle with a slide that is mounted such that it can be moved along a longitudinal axis of the surgical instrument and is connected to the surgical tool. If an instrument, such as an electrode, is inserted into the working element, the cutting loop of the electrode can be moved in the axial direction by moving the carriage axially—for example, to ablate tissue.

Different instruments, such as electrodes, laser fibers, or the like, can be inserted into the surgical instrument. To use the instrument within the surgical instrument, the carriage must be coupled to the instrument, so that the instrument can be moved by the movement of the carriage within the surgical instrument.

The use of different instruments within a surgical instrument, which requires the installation and removal of different instruments, takes time.

### SUMMARY

It is an object of the invention to optimize the use of at least two instruments within a surgical instrument.

According to the invention, this object is achieved by a carriage device having features according to the invention and/or by a surgical instrument having features according to the invention.

Accordingly, the following are provided:

A carriage device for a surgical instrument with at least two instruments, with a carriage base, a kinematic unit configured for actuation, with a first carriage module configured for coupling to a first instrument, and with a second carriage module configured for coupling to a second instrument, wherein the two carriage modules are movable relative to each other and can be individually coupled to the kinematic unit in order to use the first instrument or the second instrument, and wherein the first carriage module and the second carriage module can be fixed to the carriage base when they are not in use. A surgical instrument with two instruments also includes a carriage device. The instruments can be coupled to the kinematic unit independently of each other via the carriage modules.

The finding underlying the present invention is that a carriage can be coupled to different instruments of a surgical instrument without installing or removing the instruments.

The idea underlying the present invention is to design the carriage to be multi-part, thereby providing at least two carriage modules. Each of these carriage modules is coupled to an instrument. If an instrument is to be used, the corresponding carriage module is coupled to a kinematic unit of the surgical instrument. The instrument can then be used as usual. If the other instrument is to be used, the other carriage module can be coupled to the kinematic unit. A modular, in particular split, carriage structure is therefore provided.

In other words, the instruments can be coupled independently or selectively to the kinematic unit. The carriage module can also be configured for more than two different instruments or can be divided into a plurality of parts, wherein each of the instruments can be coupled independently to the kinematic unit. Thus, more than two carriage modules can also be provided, each of which can be configured as described.

The kinematic unit can, for example, form part of the carriage itself. Furthermore, a thumb ring, a handle, or the like can be arranged on the kinematic unit, so that the corresponding carriage module can be moved back and forth.

The kinematic unit is configured for actuation. This means in particular that the kinematic unit can be moved along a longitudinal direction of the surgical instrument, which direction in particular forms a predetermined axis. The kinematic unit can therefore be used, in particular, to control the movement of the instruments.

The carriage base can in particular be arranged on the carriage device on a side opposite a distal end of the surgical instrument. Preferably, the carriage base is arranged on a side surface of the carriage device. In particular, the carriage base can form a side surface of the carriage device. The carriage base can be configured as a flat element, in particular as a plate element.

The kinematic unit can be arranged adjacently to the carriage base. The kinematic unit can comprise a portion of a housing, a thumb ring, or other elements that are moved along a longitudinal axis of the surgical instrument when an instrument is in use. Preferably, the kinematic unit can be moved away from the carriage base, wherein the kinematic unit can also move the carriage modules away from the carriage base. This allows the selected instrument to be used.

The carriage base is configured in particular as a stationary carriage base. This means in particular that the carriage base is stationary, while the kinematic unit can move or shift relative to the carriage base. Since the carriage modules can be coupled to the kinematic unit, the carriage modules can be shifted or moved relative to the stationary carriage base.

The first carriage module is configured to be coupled to a first instrument. This means in particular that the first instrument can be fixed to the first carriage module. This can be done, for example, via at least one guide sleeve which is fixed to the first carriage module and in which the first instrument can be guided. Alternatively or additionally, fastening means may be provided with which the first instrument is fastened to the first carriage module. A movement of the first carriage module can therefore be transferred directly to the first instrument. The representations made also apply to the second carriage module with the second instrument.

The instruments can be electrodes, laser fibers, or the like. For example, an electrode, also in combination with a needle, especially for injecting tissue, can be combined with a laser fiber in a surgical instrument.

The surgical instrument may be a resectoscope. Use with other surgical instruments is also conceivable.

Advantageous embodiments and developments are disclosed in the claims and in the description with reference to the figures in the drawings.

According to an advantageous embodiment, only one of the carriage modules can be coupled at a time. This allows one instrument to be selected at a time, whereas at least one further instrument can be stored in the surgical instrument and does not interfere with the use of the other instrument. In particular, the instruments can be arranged parallel to each other and/or in holders. In this way, each instrument can be guided in a kind of sleeve, wherein the holders can be coupled to and moved with the carriage module. The carriage module can be coupled to the kinematic unit, for example, by at least one mechanical or magnetic connecting means, in particular by a positive or non-positive connection between the kinematic unit and the corresponding carriage module. Whereas one carriage module is coupled to the kinematic unit, all other carriage modules are preferably not coupled to the kinematic unit. Therefore, a desired instrument, such as a resectoscope, can be selected for use with the surgical instrument.

According to an advantageous embodiment, the kinematic unit can be configured to move the carriage modules along a predetermined axis. The predetermined axis runs in particular in the longitudinal direction of the surgical instrument. In particular, the predetermined axis is aligned parallel to the guide of the instruments. In particular, each carriage module can move axially in the longitudinal direction of the surgical instrument.

According to an advantageous embodiment, the first carriage module and the second carriage module can be fixed selectively, individually or jointly to the carriage base. In particular, the carriage base can contact and/or fix the at least two carriage modules independently of each other. For example, a plurality of recesses can be provided in the carriage base, wherein a carriage module can be fixed in each recess. This can be done, for example, using a retention device (retention means). Furthermore, magnetic regions can be provided on the carriage base, so that the retaining elements can be magnetically held on the carriage base at desired positions.

According to an advantageous embodiment, each carriage module can therefore have a retention device (retention means) configured to hold the corresponding carriage module on the carriage base. Advantageously, each carriage module can then be held separately on the carriage base or detached therefrom. The corresponding carriage module or instrument can be selected via an actuating element.

The retention means can be a locking element, an eyelet element, a spreading element, or the like. Furthermore, the retaining element can be at least partially magnetic, so that it can be held on the carriage base by a magnetic force—for example, by means of electromagnetic coupling.

According to an advantageous embodiment, the retention means can effect an axial locking in a movement direction of the corresponding carriage module. The direction of movement can coincide with the predetermined axis. Advantageously, each carriage module can, on one side surface, be in contact with the carriage base and/or fixed thereto. If the axial locking is released, the carriage modules can therefore be removed independently of one another from the carriage base. The carriage module with the unused instrument can remain fixed to the carriage base by the separate axial locking.

According to an advantageous embodiment, in a force-free initial state, both carriage modules can be held on the carriage base by the corresponding retention means. In other words, this can provide a so-called locked initial state in which none of the instruments can move away from the carriage base. This provides a safeguard that can prevent unintentional use of the instruments as well as damage to the instruments, in particular due to being pulled out from the instrument shaft in an uncontrolled manner.

According to an advantageous embodiment, the retention means can be configured to be self-resetting. In particular, each retention means can have a spring element. For example, a spring element can be mounted in a guide groove in such a way that it acts as a compression spring against a corresponding retention means. The self-resetting effect ensures that, in an unloaded initial state, the carriage modules are locked to the carriage base, thus preventing the instruments from being pulled out of the surgical instrument in an uncontrolled manner.

According to an advantageous embodiment, an actuating element can be provided for releasing the first or the second carriage module from the carriage base. The actuating element is arranged in particular laterally on the carriage device, so that it can be easily reached by an operator during use of the surgical instrument. The actuating element is preferably arranged such that it can be reached with a finger when an operator holds the surgical instrument by a handle.

Advantageously, the actuating element has a dual function. The actuating element can, on the one hand, release a carriage module from the carriage base and, on the other, couple the same carriage module to the kinematic unit. This advantageously creates functional integration. For this purpose, the actuating element can form a positive coupling with the corresponding carriage module in order to transfer the forces from the kinematic unit to the carriage module. The actuating element can also transmit force from the kinematic unit to the carriage module and thus to the instrument, so that the movement of the kinematic unit can be transferred to the instrument.

According to an advantageous embodiment, the actuating element can be configured as a mechanical slider or as an electromagnetic switch. The actuating element can therefore be understood as an operating element that moves mechanically or as an actuator that can be controlled electromagnetically. The slider or switch can be in contact with one of the carriage modules in order to release it from the carriage base. By moving the slider or switch back and forth, the corresponding instrument can be selected. For example, locking positions can be provided for the slider or switch in order to easily select a corresponding position, even without an operator having to look at the switch or slider.

The actuating element can also be configured as a type of eccentric or lever, which can in particular perform a rotational or pivoting movement. The eccentric or lever can also be controlled and operated with just one finger of one hand. In particular, this can be pivoted by up to 180°, or even up to 360°, in order to release the first or the second carriage module from the carriage base and couple it to the kinematic unit. In particular, the lever or eccentric can be moved from a first vertical orientation for coupling to the first carriage module, into a horizontal orientation for releasing both carriage modules, and into a second vertical orientation for coupling to the second carriage module.

The lever can have a guide extension with a projection, wherein the projection can be configured for contacting the first or the second carriage module, in particular by rotation of the lever. The first carriage module and the second carriage module can each have a recess in which the projection can engage. This allows each carriage module to be selectively coupled to the kinematic unit using the lever.

The same features as those mentioned above for the slider also apply to the lever.

In a further embodiment, the actuating element can be provided as a remote-controlled operating unit. This can also be achieved from a position at a distance from the surgical instrument.

According to an advantageous embodiment, the actuating element can be movable transversely to the predetermined axis. This can prevent the operation of the actuating element from triggering a movement of the instrument in the axial direction of the surgical instrument. Such a direction of movement can also be formed by a toggle switch that can be tilted about the predetermined axis.

According to an advantageous embodiment, the actuating element can have a guide extension which can be guided in a corresponding guide groove of the first and second carriage modules. The retention means can also be mounted in the guide groove, so that the actuating element, in particular the guide extension, can directly contact each retention means. By contact of the guide extension with a retention means, said retention means can be moved in a targeted manner within the guide groove, whereby the retention means can be released from or fixed to the carriage base, and coupling to the kinematic unit can be achieved.

According to an advantageous embodiment, the guide groove can be aligned transversely to the predetermined axis. In particular, the alignment of the guide groove determines the direction of movement of the actuating element.

Preferably, the guide groove is also formed in a housing of the carriage device, wherein the operating element can be arranged on the outside of the housing and is guided through the guide groove with the guide extension in order to contact the two carriage modules or the retention means. The housing can also be part of the kinematic unit and move at least partially with the corresponding carriage module.

According to an advantageous embodiment, the actuating element for releasing the retention means can be configured to release from the carriage base one of the carriage modules and to couple it to the kinematic unit. For example, the actuating element can be used to release a positive connection between a retention means and the carriage base.

According to an advantageous embodiment, the retention means can each have a locking lug, wherein, by moving the actuating element, one of the locking lugs is released from the carriage base, and the other locking lug remains in engagement with the carriage base. Such a positive locking connection can be achieved by means of the locking lug. Each locking lug can be guided through a recess in the carriage base and engage with an undercut in the carriage base. To release the locking lug from the undercut, the corresponding retention means can be moved in the guide groove, whereby the locking lug can be moved within the recess in the carriage base. This allows the locking lug to be guided out of the recess, whereby the carriage module can be released from the carriage base.

According to an advantageous embodiment, the carriage modules can be mounted in a multi-part housing, wherein a first housing part forms the carriage base, and a second housing part forms part of the kinematic unit. For example, a housing side wall that is located furthest from the distal end of the surgical instrument can form the carriage base. The rest of the housing, which together with the housing side wall that forms the carriage base can completely enclose a volume, can form part of the kinematic unit. The housing can completely enclose the carriage modules and, for example, have a cuboid shape.

The above embodiments and developments can be combined with each other as desired, if appropriate. Further possible embodiments, developments, and implementations of the invention also include combinations, which are not explicitly mentioned, of features of the invention described above or below with respect to the exemplary embodiments. In particular, a person skilled in the art will also add individual aspects as improvements or additions to the particular basic form of the present invention.

The present invention is explained in greater detail below with reference to the exemplary embodiments shown in the schematic figures of the drawing. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a side view of a surgical instrument with a carriage device;

FIG. 2 is a detail view of FIG. 1 in an isometric representation;

FIG. 3 is a further detail view from FIG. 1 in a side view;

FIG. 4 is a detail view of a carriage device;

FIG. 5a is a sectional view through an embodiment of a carriage device;

FIG. 5b is a sectional view through a further embodiment of a carriage device;

FIG. 6 is a further sectional view through an embodiment of a carriage device;

FIG. 7 is a detail view of a carriage device;

FIG. 8a and FIG. 8b are two detail views of an actuating element;

FIG. 9a is a further side detail view of a carriage device in one of two states;

FIG. 9b is a further side detail view of a carriage device in another of two states;

FIG. 10 is a side view of a surgical instrument with a carriage device;

FIG. 11 is a detail view from FIG. 10 in a perspective representation;

FIG. 12 is a further detail view from FIG. 10 in a side view;

FIG. 13 is a detail view of the carriage device from FIG. 10;

FIG. 14 is a sectional view through the embodiment of the carriage device from FIG. 10;

FIG. 15 is a further sectional view through the embodiment of the carriage device from FIG. 10;

FIG. 16 is a detail view of the carriage device from FIG. 10; and

FIG. 17 is a detail view of an actuating element in a possible embodiment.

The accompanying figures of the drawing are intended to provide a further understanding of the embodiments of the invention. They illustrate embodiments and, in conjunction with the description, serve to explain principles and concepts of the invention. Other embodiments and many of the advantages mentioned are shown in the drawings. The elements in the drawings are not necessarily shown to scale.

In the figures of the drawing, identical, functionally equivalent, and identically acting elements, features, and components are each provided with the same reference signs, unless otherwise specified.

### DESCRIPTION OF PREFERRED EMBODIMENTS

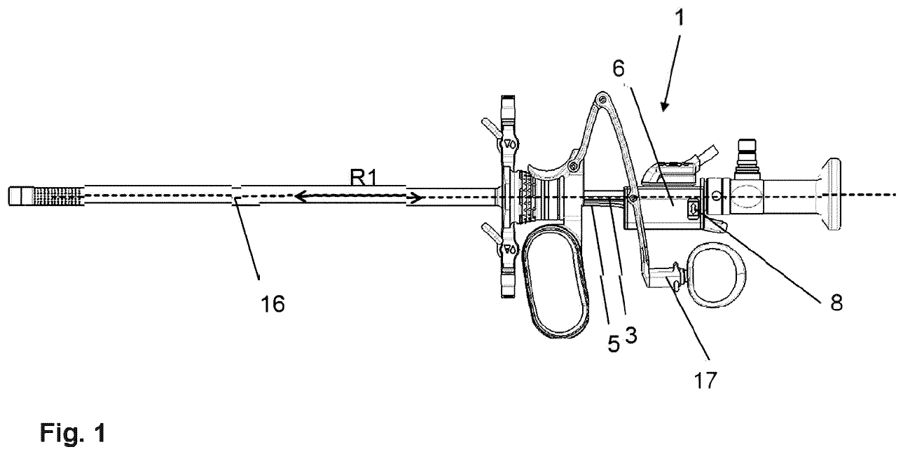

Referring to the drawings, FIG. 1 shows a side view of a surgical instrument with a carriage device 1. Two instruments 3, 5 are arranged in the surgical instrument, wherein both instruments are coupled to the carriage device 1. In the side view shown, only an external view of the carriage device 1 is visible, wherein a housing of the carriage device 1 can form part of a kinematic unit 6. Furthermore, parts of the handling device, such as a thumb ring 17, are arranged on the kinematic unit 6. The carriage device 1 can move on a predetermined axis R1 in the direction of the axis R1, whereby a first instrument 3 or a second instrument 5 can be moved in an instrument shaft 16. In order to select the desired instrument 3, 5, an actuating element 8 is provided which is arranged on the housing of the carriage device 1.

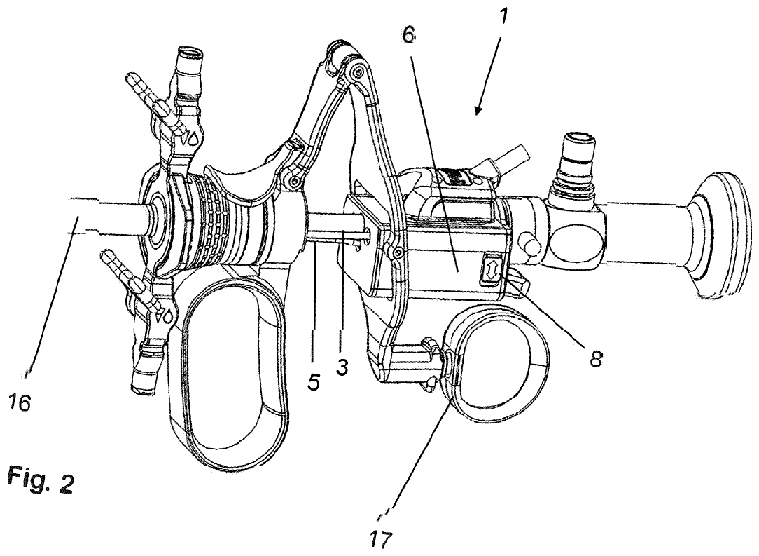

FIG. 2 shows a detail view of FIG. 1 in an isometric representation. Here, for one, the instruments 3, 5 can be seen, which are guided out of the housing of the carriage device 1. The instruments 3, 5 can be guided in sockets, wherein the socket moves in particular with the corresponding instrument 3, 5. The direction in which the actuating element 8 can move is indicated in the direction of the arrow on the actuating element 8. In this embodiment, the actuating element 8 is configured as a type of slider and can be moved upwards or downwards in the illustration.

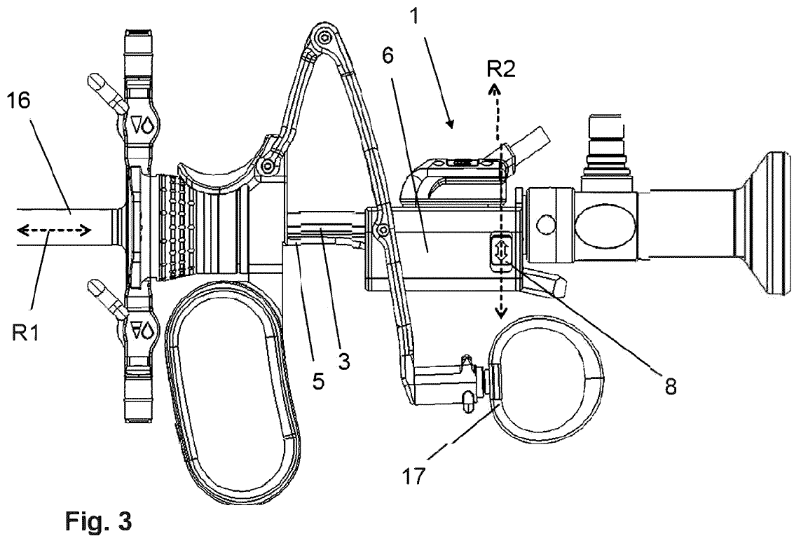

FIG. 3 shows another detail view from FIG. 1 in a side view. In this view, the direction of movement is shown on the R2 axis of the slider, which is oriented transversely to a direction of movement along the R1 axis. By moving the actuating element 8, an uncontrolled movement of an instrument 3, 5 out of the instrument shaft 16 can therefore be prevented.

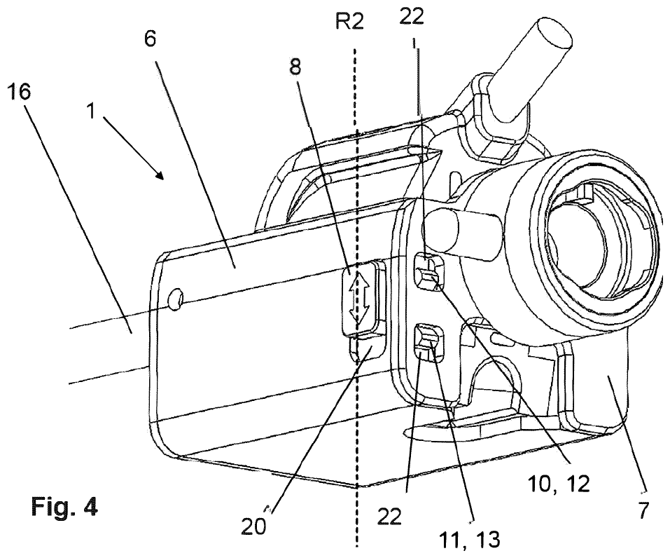

FIG. 4 shows a detail view of a carriage device 1. Here, a side surface of the carriage device 1 can be seen which is arranged opposite a distal end of the surgical instrument. The side surface can, for example, be configured as a carriage base 7, in particular a stationary carriage base 7, and, in particular, form a side surface of the carriage device 1. In the carriage base 7, two recesses 22 can be seen, in each of which a retention deice (retention means) 10, 11 is arranged. The retention means 10, 11 engage with a corresponding locking lug 12, 13 through the recess 22 and can hook into an undercut in the recess 22. This makes it possible to create a locked state, particularly in a force-free initial state. In such a state, both instruments 3, 5 are anchored to the carriage base 7, so that each instrument 3, 5 can be prevented from slipping out of the instrument shaft 16. Only by moving the actuating element 8 can a first instrument 3 or a second instrument 5 be selected and coupled to the kinematic unit 6. For this purpose, the actuating element 8 can also be used in particular to transmit force from the kinematic unit 6 to the selected carriage module.

As an alternative to a locking lug, the retention means 10, 11 can also have a fixing element, in particular one without locking, which is configured, for example, as a type of eyelet. Furthermore, for example, a spreading element can be used which anchors itself to the carriage base 7 by spreading.

In an embodiment not shown, the retaining elements 10, 11 can also be configured as magnetic retaining elements which hold the corresponding carriage module 2, 4 to the carriage base 7 by means of a magnetic force. In such an embodiment, the carriage base 7 can also be configured without recesses 22 and in particular can have a magnetic material at least in some regions.

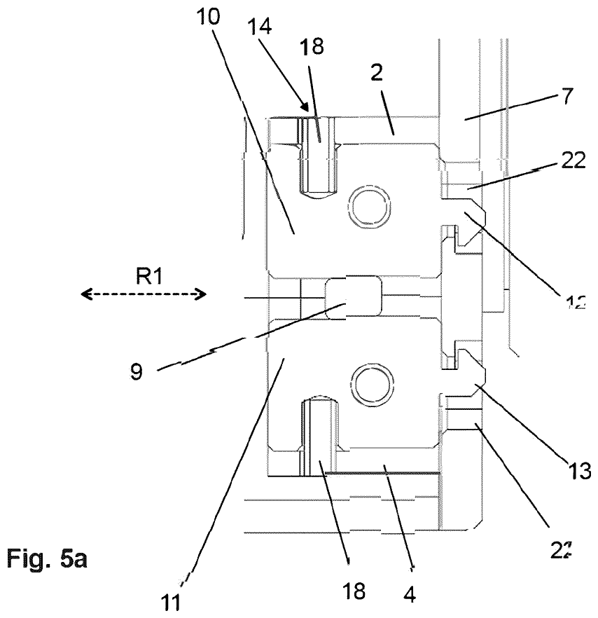

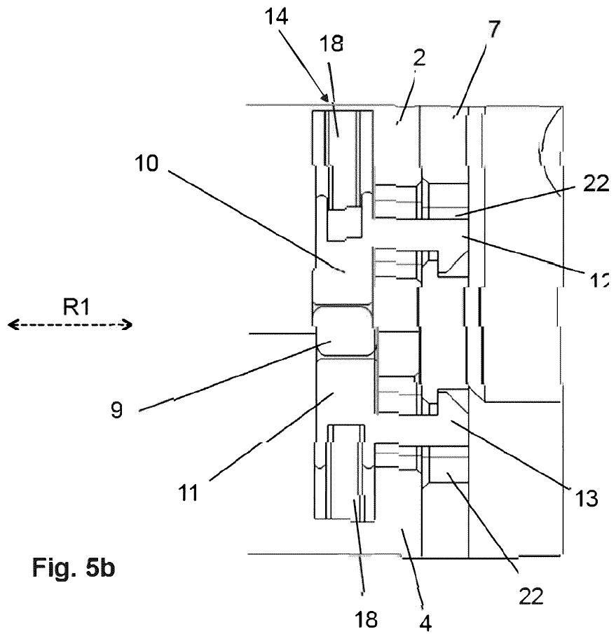

FIG. 5a and FIG. 5b each show a sectional view through an embodiment of a carriage device 1. Each sectional view shows a longitudinal section through the carriage device 1, wherein only a partial region is shown. In this partial region, the carriage base 7 can be seen, which has two recesses 22. A retention means 10, 11 is held in each of the recesses 22, wherein a corresponding locking lug 12, 13 of each retention means 10, 11 engages in the recess 22. For this purpose, an undercut is provided in the recess 22, in which the locking lugs 12, 13 can be positively engaged. The depicted situation shows a force-free initial state in which the carriage device 1 is locked. The locking can be achieved, for example, by the two retention means 10, 11 being configured to be self-resetting. In the embodiments shown, spring elements 18 can be provided, wherein each spring element 18 acts upon a retention means 10, 11 as a compression spring. In a force-free initial state, the spring elements 18 therefore press against the retention means 10, 11, wherein the retention means 10, 11 are pressed in the direction of a guide extension 9 of the actuating element 8. If the guide extension 9 is moved upwards or downwards in the illustration, i.e., transversely to the predetermined axis R1, a desired retention means 10 or 11 can be moved. This allows the retention means 10 or 11 to detach from the carriage base 7, thereby releasing the carriage module 2 or the carriage module 4. This allows a desired carriage module 2 or 4 to be coupled to the kinematic unit 6.

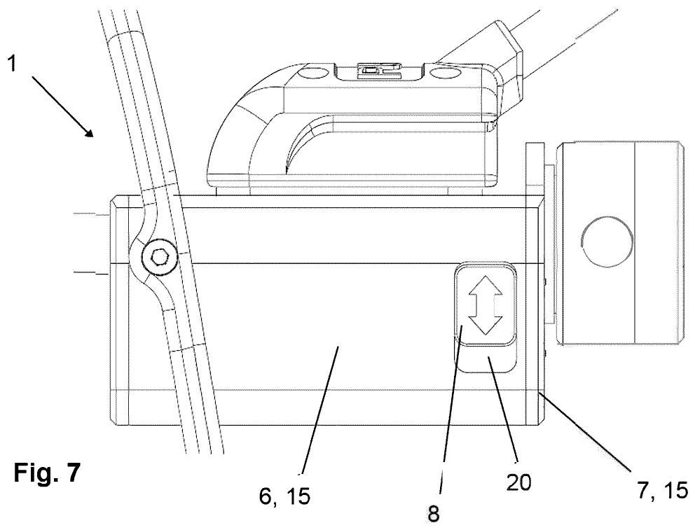

The spring elements 18 can be guided in a guide groove 14. As shown in FIG. 5b, the guide extension 9 can also be guided in the guide groove 14. Furthermore, the guide extension 9 can be guided offset from the spring elements 18 in a separate guide groove, as shown by way of example in FIG. 5a. The guide groove can also be formed in the housing 15, e.g., shown in FIG. 7, so that the guide extension 9 can pass from an outer side of the housing 15 into the interior of the housing 15 in order to contact or move the carriage modules 2, 4 or the retention means 10, 11.

In one possible embodiment, each carriage module 2, 4 can therefore be coupled to the kinematic unit 6 via the actuating element 8 and a corresponding retention means 10, 11, wherein these means are guided in a guide groove 14 and can be moved along this guide groove 14. Other configurations, in particular with a magnetic holder or storage means or the like, may be provided.

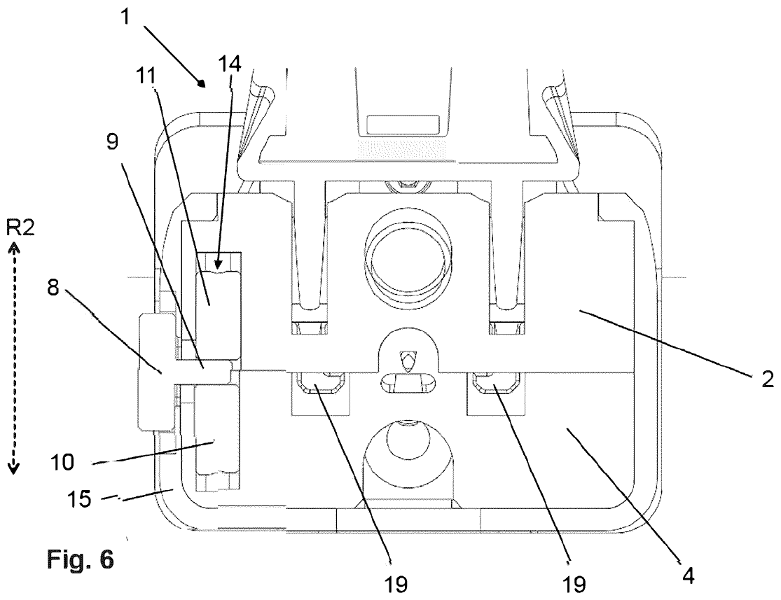

FIG. 6 shows a further sectional view through an embodiment of a carriage device 1. The sectional view shows a cross-section, wherein this passes through the actuating element 8. The shape of the actuating element 8 can be seen, wherein the guide extension 9 engages in the interior of the housing 15.

The first carriage module 2 and the second carriage module 4 are arranged one above the other, so that they can be moved separately and independently of each other. The guide groove 14 runs partially in the first carriage module 2 and in the second carriage module 4. In a force-free initial state, the guide extension 9 is located in particular at the height of the contact surfaces at which the first carriage module 2 and the second carriage module 4 contact each other. As a result, one of the carriage modules 2, 4 can be coupled to the kinematic unit 6 by means of a minimal movement of the actuating element 8. The guide extension 9 can therefore form a positive connection with the corresponding carriage module in order to transfer forces from the kinematic unit 6 to the carriage module decoupled from the carriage base 7.

Additional recesses and further components, such as an RF connector 19, can be integrated into the carriage modules 2, 4. Furthermore, recesses can be provided which serve to stabilize the movement of the corresponding carriage module 2, 4.

FIG. 7 shows a detail view of a carriage device 1. In the embodiment shown, the actuating element 8 is configured as a slider, wherein the slider is shown pushed upwards within the recess 20. As a result, the carriage module arranged at the top in this embodiment and the associated instrument can be coupled to the kinematic unit 6. In this embodiment, the kinematic unit 6 comprises a part of the housing 15. The carriage base 7 forms another part of the housing 15. In this way, a complete housing 15 can be formed which can enclose a volume. A part of the housing 15 can therefore move with the kinematic unit 6 when one of the instruments is in use. The carriage base 7 remains immobile in the position, so that the second instrument, which is fixed to the carriage base 7, does not move.

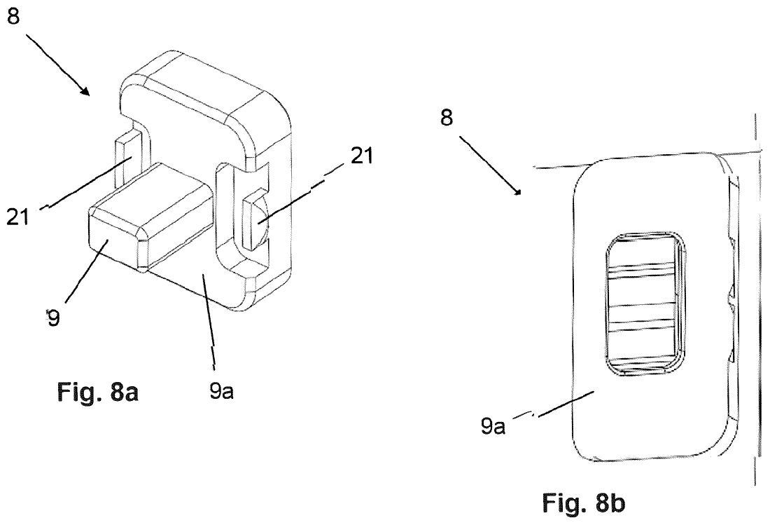

FIG. 8a and FIG. 8b show two detail views of an actuating element 8. The slider shown has the guide extension 9, which is arranged protruding from an engagement surface 9a. The engagement surface 9a can be reached by an operator with a finger in order to push the actuating element 8 into the desired position. For this purpose, for example, guides 21 can be provided which allow a secure guidance of the actuating element 8 in the guide groove 14, as shown in FIG. 8a. In FIG. 8b, the engagement surface 9a can be seen, which can be reached by an operator. This can have a contoured, e.g., grooved, surface in order to form a frictional connection with a finder of the operator.

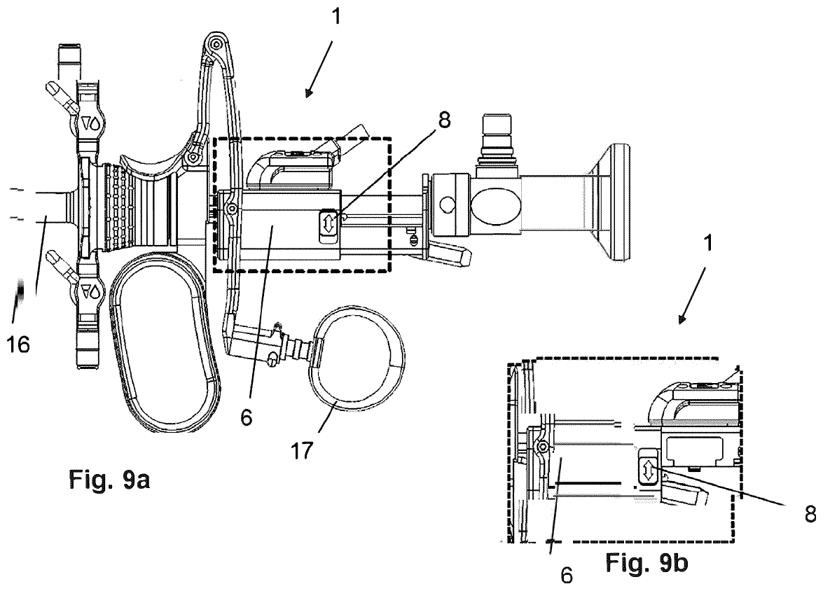

FIG. 9a and FIG. 9b show another side view of a carriage device 1 in two states. FIG. 9a shows the state as in FIG. 7, wherein the actuating element 8 is shown moved upwards. Consequently, the carriage module arranged in particular at the top can be coupled to the kinematic unit 6, whereas the at least one further carriage module remains anchored to the carriage base 7. FIG. 9b shows a further state in which the actuating element 8 is shown moved downwards. Consequently, another carriage module can be coupled to the kinematic unit, whereas the previously coupled carriage module is anchored again to the carriage base.

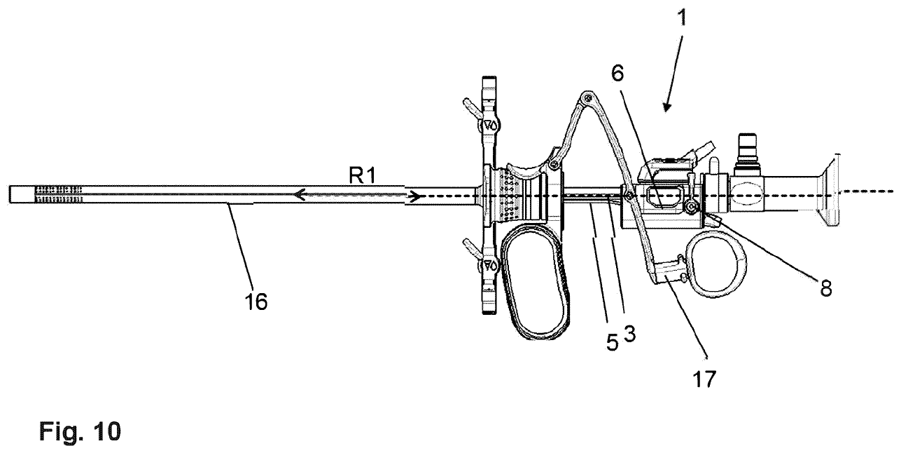

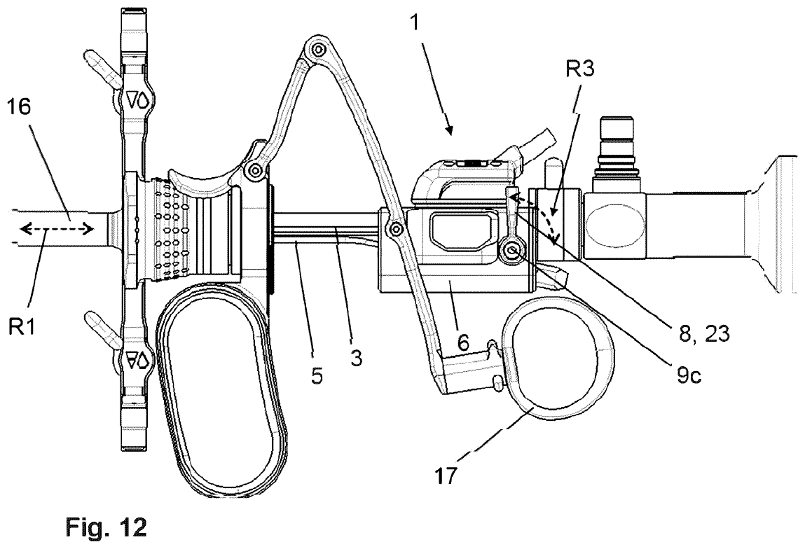

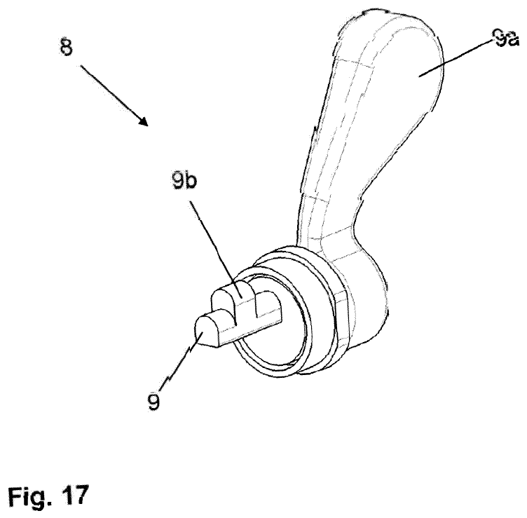

FIGS. 10 to 17 show a further possible embodiment of a carriage device 1, wherein in particular the actuating element 8 is shown in a further possible embodiment. In the further features, this embodiment may correspond to the embodiment shown in FIGS. 1 to 9, such that not every feature described above is described again in detail.

FIG. 10 shows a side view of a surgical instrument with a carriage device 1 in such a further embodiment. The actuating element 8 is configured as a lever or eccentric, wherein the lever or eccentric can be rotated about an axis or, in particular manually, pivoted in order to couple one of the carriage modules to the kinematic unit. The actuating element 8 can, on the one hand, release a carriage module 2, 4 from the carriage base 7 and, on the other hand, couple the same carriage module 2, 4 to the kinematic unit 6. The kinematic unit 6 can comprise a portion of a housing 15, a thumb ring 17, or the like.

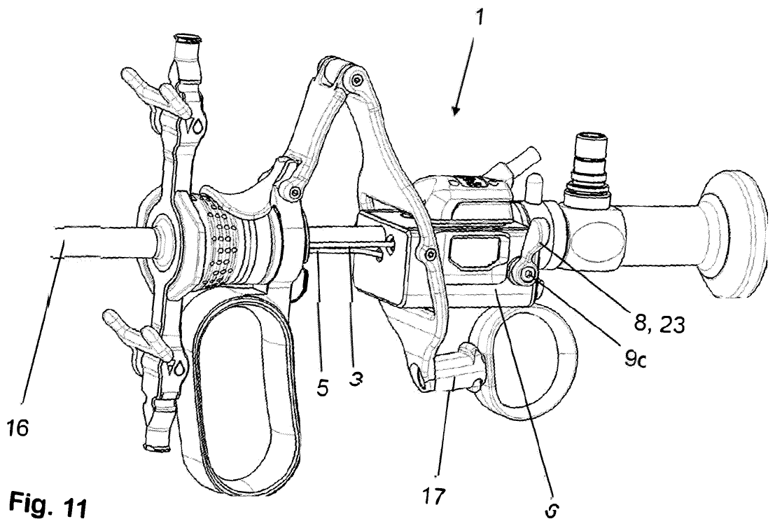

FIGS. 11, 12 and 16 each show a detail view from FIG. 10 in a perspective representation or a side view. In this embodiment, the actuating element 8 has a rotation axis 9c and a lever element 23 arranged thereon, wherein the lever element 23 forms at least one engagement surface 9a. In particular, two opposing engagement surfaces 9a are provided on the lever element 23, so that the lever can be rotated in opposite rotation directions R3 by pressure, in particular manual pressure with a finger against the lever element 23.

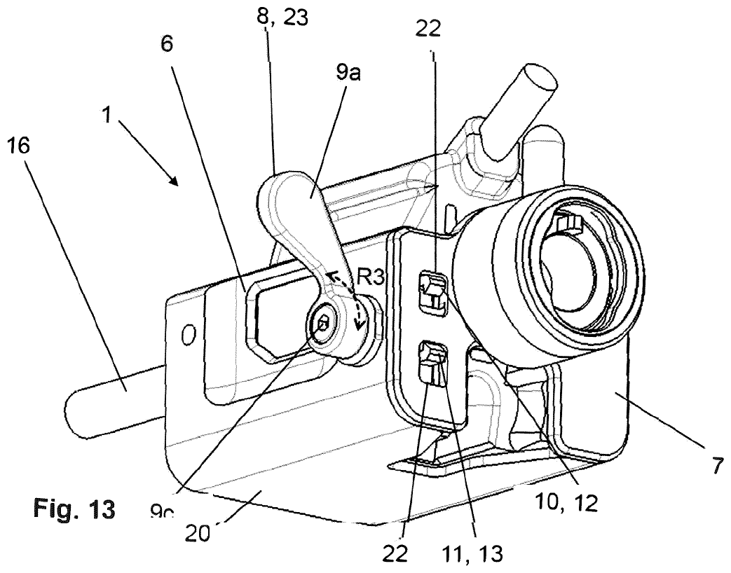

FIG. 13 shows a detail view of the carriage device 1 from FIG. 10. By rotating the lever about the rotation axis 9c, one of the carriage modules 2, 4 can be released from the carriage base 7, whereby the lever element 23 can be rotated in the rotation direction R3. Thus, the lever element 23 can be pivoted in particular by an angle of up to 180° in order to release the first or the second carriage module 2, 4. The illustration shows the lever or the lever element 23 in a first vertical orientation for coupling to the first carriage module 2. If the lever or lever element 23 is pivoted to the right, it can be brought into a horizontal orientation for releasing both carriage modules 2, 4. The lever or lever element 23 can then be pivoted downwards and brought into a second vertical orientation for coupling to the second carriage module 4.

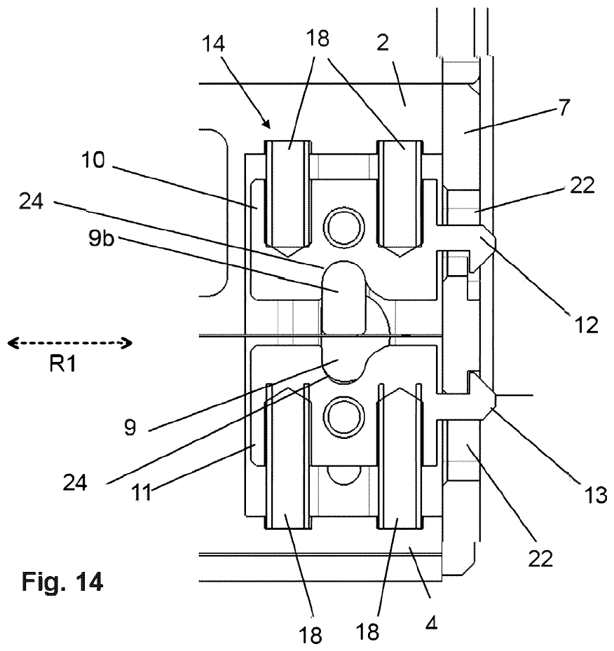

FIG. 14 shows a sectional view, wherein the lever or the lever element 23 is shown vertically oriented as in FIG. 13. In such a configuration, the first carriage module 2 is released from the carriage base 7, wherein the locking lug 12 is shown released from the recess 22 in the carriage base. This is achieved in particular by a projection 9b which protrudes from the guide shoulder 9. By rotating the lever and thus rotating the guide extension 9, the projection 9b can be brought to a different peripheral position, wherein either the first carriage module 2 or the second carriage module 4 can be contacted. The first carriage module 2 and the second carriage module 4 each have for this purpose a recess 24 in which the projection can engage.

In contrast to the illustration in FIGS. 5a and 5b, two spring elements 18 are also provided. This can advantageously compensate for asymmetrical loading. A design with only one spring element 18 is also conceivable.

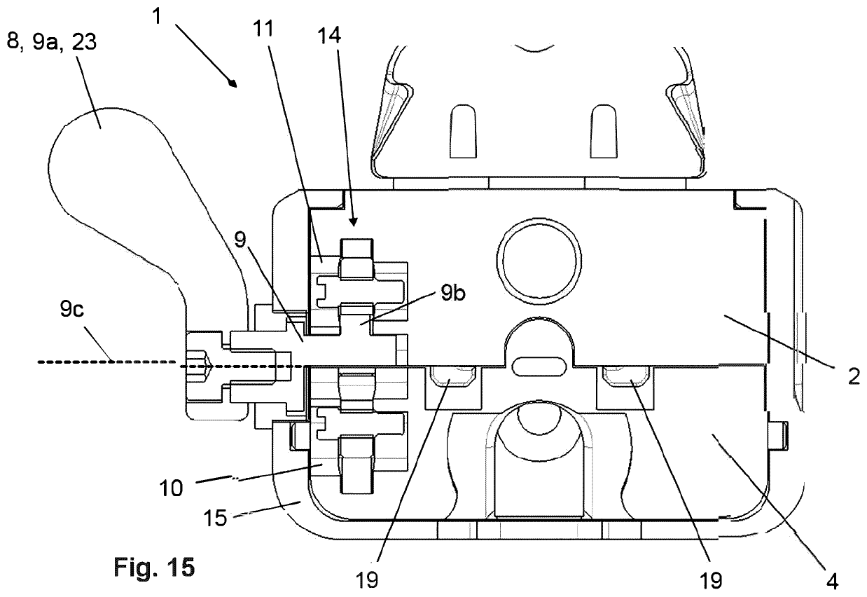

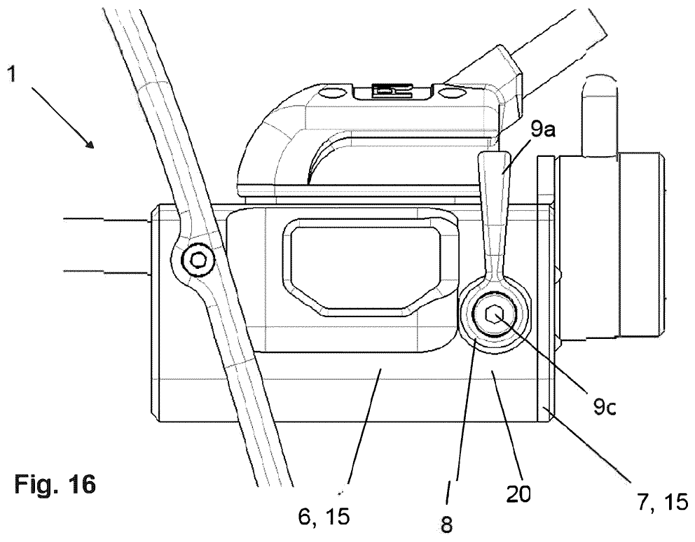

FIG. 15 shows a sectional view through the embodiment of the carriage device 1 as shown in FIG. 14. In this embodiment, the guide extension 9 has a projection 9b. The projection 9b is provided in a partial region of the guide extension 9 in order to couple one of the carriage modules 2, 4 to the kinematic unit 6 during a rotational movement of the guide extension 9.

The projection 9b is configured in particular as a radial projection, so that it can be oriented in different radial directions with respect to the guide extension 9a by rotating the lever.

The projection 9b can also be formed at one end of the guide extension 9 or over the entire length of the guide extension 9. Preferably, however, the projection 9b is provided on the guide extension 9 as an asymmetrical element with respect to a rotation axis.

Preferably, the projection 9b is oriented in the same direction as the lever element 23 or the engagement surface 9a of the actuating element 8.

FIG. 17 shows a detail view of an actuating element 8 in a possible further embodiment, as this can also be used in an embodiment of a carriage device 1 according to FIGS. 10 to 16.

The guide extension 9 and the projection 9b can have different radii. Likewise, the guide extension 9 and the projection 9b can be configured with the same radius. The radius of the projection 9b is in particular adapted to a radius of a recess 24 in the relevant carriage base 2, 4, as shown in FIG. 14.

Although the present invention has been fully described above with reference to preferred exemplary embodiments, it is not limited thereto, but can be modified in a variety of ways. In particular, the number of instruments and thus the number of carriage modules can be more than two. Furthermore, the kinematic unit can have a different geometry; in particular, the housing 15 can have a geometry adapted to the carriage modules 2, 4, and possibly further carriage modules. Furthermore, the embodiments can be combined with one another to create further embodiments. In particular, the actuating element 8 according to FIGS. 10 to 17 can also be implemented with only one spring element 18, as shown in FIG. 5.

While specific embodiments of the invention have been shown and described in detail to illustrate the application of the principles of the invention, it will be understood that the invention may be embodied otherwise without departing from such principles.

### LIST OF REFERENCE CHARACTERS

1 carriage device
2 first carriage module
3 first instrument
4 second carriage module
5 second instrument
6 kinematic unit
7 carriage base
8 actuating element
9 guide extension
9a engagement surface
9b projection
9c rotation axis
10 retention means
11 retention means
12 locking lug
13 locking lug
14 guide groove
15 housing
16 shaft
17 thumb ring
18 spring element
19 RF connector
20 recess
21 guide
22 recess
23 lever element
10 R1 axis
R2 axis
R3 rotation direction

What is claimed is:

1. A carriage device for a surgical instrument with at least two instruments, the carriage device comprising:
a carriage base;
a kinematic unit configured for actuation;
a first carriage module configured to couple to a first instrument of the at least two instruments; and
a second carriage module configured to couple to a second instrument of the at least two instruments, wherein the two carriage modules are movable relative to each other and are configured to be coupled individually to the kinematic unit for use of the first instrument or the second instrument, and wherein the first carriage module and the second carriage module are configured to be fixed to the carriage base with the first carriage module and the second carriage module not in use.

2. A carriage device according to claim 1, wherein the carriage device is configured such that only one of the first carriage module and the second carriage module can be coupled to the kinematic unit at a time.

3. A carriage device according to claim 1, wherein the kinematic unit is configured to move the carriage modules along a predetermined axis.

4. A carriage device according to claim 1, wherein the first carriage module and the second carriage module are configured to be fixed selectively, individually or jointly to the carriage base.

5. A carriage device according to claim 4, wherein each of the first carriage module and the second carriage module comprises a retention device configured to hold the corresponding carriage module on the carriage base.

6. A carriage device according to claim 5, wherein the retention means effects an axial locking of the corresponding carriage module in a movement direction.

7. A carriage device according to claim 5, wherein, in a force-free initial state, both the first carriage module and the second carriage module are held on the carriage base by the corresponding retention device.

8. A carriage device according to claim 7, wherein the retention device is configured to be self-resetting, wherein each retention device comprises a spring element.

9. A carriage device according to claim 4, further comprising an actuating element configured to release the first or the second carriage module from the carriage base.

10. A carriage device according to claim 9, wherein the actuating element is configured as a mechanical slider or as an electromagnetic switch.

11. A carriage device according to claim 9, wherein the actuating element is movable transversely to the predetermined axis.

12. A carriage device according to claim 9, wherein the actuating element comprises a guide extension configured to be guided in a corresponding guide groove of the first and second carriage modules.

13. A carriage device according to claim 12, wherein the guide groove is aligned transversely to the predetermined axis.

14. A carriage device according to claim 9, wherein each of the first carriage module and the second carriage module comprises a retention device configured to hold the corresponding carriage module on the carriage base, and wherein the actuating element is configured to release the retention device so as to release one of the carriage modules from the carriage base and to couple the released one of the carriage modules to the kinematic unit.

15. A carriage device according to claim 14, wherein each retention device comprises a locking lug, wherein one of the locking lugs is released from the carriage base by movement of the actuating element, and the other of the locking lugs remains in engagement with the carriage base.

16. A carriage device according to claim 4, wherein the carriage base is comprised by a first housing part and the kinematic unit is comprised by a second housing part and the first housing part and the second housing part cooperate to form a multi-part housing, wherein the carriage modules are mounted in the multi-part housing.

17. A surgical instrument comprising:
a first instrument;
a second instrument; and
a carriage device, the carriage device comprising:
a carriage base;
a kinematic unit configured for actuation;
a first carriage module configured to couple to the first instrument; and
a second carriage module configured to couple to the second instrument, wherein the two carriage modules are movable relative to each other and are configured to be coupled individually to the kinematic unit for use of the first instrument or the second instrument, and wherein the first carriage module and the second carriage module are configured to be fixed to the carriage base with the first carriage module and the second carriage module not in use, and wherein the first instrument and the second instrument are configured to be coupled to the kinematic unit independently of each other via the carriage modules.

18. A surgical instrument according to claim 17, wherein each of the first carriage module and the second carriage module comprises a retention device configured to hold the corresponding carriage module on the carriage base, wherein the carriage device is configured such that only one of the first carriage module and the second carriage module can be coupled to the kinematic unit at a time, wherein the kinematic unit is configured to move the carriage modules along a predetermined axis.

19. A surgical instrument according to claim 18, further comprising an actuating element configured to release the first or the second carriage module from the carriage base and wherein the actuating element is configured to release the retention device so as to release one of the carriage modules from the carriage base and to couple the released one of the carriage modules to the kinematic unit.

20. A surgical instrument according to claim 19, wherein each retention device comprises a locking lug, wherein one of the locking lugs is released from the carriage base by movement of the actuating element, and the other of the locking lugs remains in engagement with the carriage base.
